SEQUENCE LISTING (6 sequences)

SEQ ID NO: 1 - DNA, 414 bp, synthetic construct
  source 1..414: mol_type = other DNA
atgagcattgaccgtacctcacctttgaaacccgttagcactgtccagacgcgcgaaaccagcgacacgccggtacaaaaaacgcgtcaggaaaaaacgtccgccgcgacgagcgccagcgtaacgttaagcgacgcgcaagcgaagctcatgcagccaggcgtcagcgacattaatatggaacgcgtcgaagcattaaaaacggctatccgtaacggtgagttaaaaatggatacgggaaaaatagcagactcgctcattcgcgaggcgcagagctacttacagagtaaaaaaggcggcggcggcagcaccagccatcatcatcatcatcatagcggcggcgaatttgcgagctggagccacccgcagttcgagaaaggcggcagcagctggagccacccgcagttcgagaaa

SEQ ID NO: 2 - DNA, 411 bp, synthetic construct
  source 1..411: mol_type = other DNA
atgagcattgaccgtacctcacctttgaaacccgttagcactgtccagacgcgcgaaaccagcgacacgccggtacaaaaaacgcgtcaggaaaaaacgtccgccgcgacgagcgccagcgtaacgttaagcgacgcgcaagcgaagctcatgcagccaggcgtcagcgacattaatatggaacgcgtcgaagcattaaaaacggctatccgtaacggtgagttaaaaatggatacgggaaaaatagcagactcgctcattcgcgaggcgcagagctacttacagagtaaaaaaggcggcagcagcaccagccatcatcatcatcatcatagcggcggcgaatttgcgagctggagccacccgcagttcgagaaaggcggcagcagctggagccacccgcagttcgagaaa

SEQ ID NO: 3 - DNA, 60 bp, synthetic construct
  source 1..60: mol_type = other DNA
ggcggcggcggcagcaccagccatcatcatcatcatcatagcggcggcgaatttgcgagc

SEQ ID NO: 4 - DNA, 57 bp, synthetic construct
  source 1..57: mol_type = other DNA
ggcggcagcagcaccagccatcatcatcatcatcatagcggcggcgaatttgcgagc

SEQ ID NO: 5 - DNA, 60 bp, synthetic construct
  source 1..60: mol_type = other DNA
tggagccacccgcagttcgagaaaggcggcagcagctggagccacccgcagttcgagaaa

SEQ ID NO: 6 - DNA, 15 bp, synthetic construct
  source 1..15: mol_type = other DNA
aggaggtttgatcct

(19) United States
(12) Patent Application Publication
(10) Pub. No.: US 2026/0028377 A1
(43) Pub. Date: Jan. 29, 2026

(54) ANTI-CANCER STRAIN EXPRESSING STREP-TAG, ANTI-CANCER COMPOSITION USING THE SAME, ANTI-CANCER ADJUVANT AND TUMOR IMAGING AGENT

(71) Applicants: THE INDUSTRY & ACADEMIC COOPERATION IN CHUNGNAM NATIONAL UNIVERSITY (IAC), Daejeon (KR); KOREA INSTITUTE OF SCIENCE AND TECHNOLOGY, Seoul (KR)

(72) Inventors: Hyo Jin LEE, Daejeon (KR); Heung Jin JEON, Daejeon (KR); Solbi KIM, Daejeon (KR); Seung Beum SUH, Seoul (KR); Sehoon KIM, Seoul (KR); Hyeon Jae LEE, Seoul (KR); Dohee LEE, Seoul (KR); Bo Geum SEO, Seoul (KR)

(73) Assignees: THE INDUSTRY & ACADEMIC COOPERATION IN CHUNGNAM NATIONAL UNIVERSITY (IAC), Daejeon (KR); KOREA INSTITUTE OF SCIENCE AND TECHNOLOGY, Seoul (KR)

(21) Appl. No.: 18/994,378

(22) PCT Filed: Jun. 29, 2023
(86) PCT No.: PCT/KR2023/009117

§ 371 Date: Jan. 14, 2025

(30) Foreign Application Priority Data
Jul. 20, 2022 (KR) 10-2022-0089823

(51) Int. Cl. C07K 14/255; A61K 35/74; A61K 49/00; A61K 51/08; C12N 1/20; C12N 15/74; C12R 1/42

(52) U.S. Cl. CPC C07K 14/255; A61K 35/74; A61K 49/0056; A61K 51/08; C12N 1/205; C12N 15/74; C07K 2319/22; C12R 2001/42

(57) ABSTRACT

The present invention relates to a gene construct characterized by encoding a fusion protein of flgM and Strep-tag through the linkage of an anti-sigma factor flgM gene and a gene encoding Strep-tag, enabling the expression of Strep-tag on the surface of an anti-cancer strain that can target tumor sites and stimulate immune activity, and to an anti-cancer adjuvant and tumor imaging agent that, when transformed by this construct, exhibits anti-cancer activity itself and can be effectively utilized in the diagnosis and treatment of tumor cells together with anti-cancer substances or contrast agents labeled with substances specifically interacting with Strep-tag.

### CROSS-REFERENCE TO PRIOR APPLICATIONS

This Application is a National Stage Patent Application of PCT International Application No. PCT/KR2023/009117 (filed on Jun. 29, 2023), which claims priority to Korean Patent application No. 10-2022-0089823 (filed on Jul. 20, 2022), which are all hereby incorporated by reference in their entirety.

### SEQUENCE LISTING

This application contains a Sequence Listing submitted via XML file and hereby incorporated by reference in its entirety. The sequence listing is named “782-0018_Sequence_Listing.xml”, created on Jan. 13, 2025 and 7,445 bytes in size.

### BACKGROUND

The present invention relates to a gene construct for expressing Strep-tag to be expressed on the surface of and/or secreted from an anti-cancer strain capable of targeting tumor sites and stimulating immune activity, and to an anti-cancer adjuvant and tumor imaging agent that, when transformed by this construct, exhibits anti-cancer activity itself and can be effectively utilized in the diagnosis and treatment of tumor cells together with anti-cancer substances or contrast agents labeled with substances, which specifically interacts with Strep-tag.

Cancer is a disease that threatens life by destroying normal cells as cell proliferation activity does not stop and invades surrounding tissues. As the body's regulatory ability decreases due to aging, it becomes vulnerable to cancer, and in modern aging society, cancer has ranked first in overall causes of death since 2007, exceeding twice the mortality rate of heart disease, which ranks second.

While strengthening immunity is the most effective response system for treating diseases, cancer cells evade the body's immune response because they are not external invaders. Certain viruses or bacteria infect cancer cells more than normal cells, and infected bacteria can become targets of immune cells. Accordingly, anti-cancer treatment methods have been proposed that stimulate the body's immune response to combat cancer cells by deliberately infecting with certain viruses or bacteria. However, infectious bacteria such as Shigella, Vibrio cholerae, and pathogenic E. coli only infiltrate intestinal cells and do not reach important organs that cause immune responses such as the liver and spleen. In contrast, Salmonella can stimulate systemic immune responses by infiltrating the spleen and liver through lymph nodes. Specifically, Leschner et al. (J. Mol. Med. 2010, 88, 763-773) traced the infection route in mice with CT26 tumors over time after intravenous injection of fluorescent Salmonella. The results showed that immediately after infection, the entire body was infected through the mouse's blood, Salmonella accumulated in the spleen and liver 20 minutes after infection, and after 24 hours, Salmonella was observed to be concentrated only in tumor tissue. This indicates that Salmonella may be more effective in anti-cancer immunotherapy compared to other strains, as it stimulates systemic immune responses in the spleen and liver and subsequently targets cancer to activate localized immunity.

However, Salmonella is a representative bacterium that causes food poisoning, and if infected in tissues other than tumors, it can threaten life by causing sepsis due to abnormal host responses. A research team at Yale University in the United States reported that genetic manipulation of Salmonella can attenuate toxicity while maintaining tumor-attacking properties, and that injection of the attenuated Salmonella can suppress tumors by inducing immune stimulation. Accordingly, extensive research is underway to develop attenuated Salmonella strains engineered to express substances with anti-cancer properties. However, attenuated Salmonella exhibits low survival and reduced immunogenicity, and there is a risk that mutations could revert attenuated strains to wild-type Salmonella, leading to sepsis, necessitating additional safety measures.

To complement the low immunogenicity of attenuated Salmonella, studies have been conducted to develop Salmonella strains that can additionally express or suppress substances that help anti-cancer treatment, and to use them for anti-cancer treatment. Patent No. 10-0852687 discloses a Salmonella strain expressing tumor necrosis factor alpha with a tumor necrosis factor alpha protein vector introduced into an attenuated Salmonella strain and an anti-cancer therapeutic composition containing the same, and Patent No. 10-1750007 discloses a solid cancer therapeutic agent using an attenuated Salmonella strain in which L-asparaginase can be selectively expressed at the tumor site. Although the above Salmonella strains additionally complemented anti-cancer activity by expressing anti-cancer substances, they still carry the risk of causing sepsis by converting attenuated strains to wild-type Salmonella. In addition, when expressing anti-cancer substances in vivo, the Salmonella strain itself may be killed by the physiological activity of the anti-cancer substances, so simultaneous for expression of anti-cancer substances and Salmonella's anti-cancer efficacy, the Salmonella strain should be able to stably survive after targeting the tumor site and continuously express and secrete anti-cancer substances.

Strep-tag is a synthetic peptide consisting of 8 amino acids that exhibits strong binding affinity toward avidin, streptavidin, or processed streptavidin, known as strep-tactin, and can be fused to the N- or C-terminus of recombinant proteins. Using the specific interactions between Strep-tag and strep-tactin, proteins containing Strep-tag can be isolated from cell lysates in a single step, making it useful for the detection and purification of specific proteins. However, there have been no reports of anti-cancer strains expressing Strep-tag capable of utilizing the specific binding affinity of Strep-tag in vivo.

### SUMMARY

The present invention aims to solve the problems of the prior art by providing a gene construct that enables the stable expression of Strep-tag on the surface of a strain and/or its secretion.

Another object of the present invention is to provide an anti-cancer recombinant strain, which is transformed by the above construct to express Strep-tag on its surface and/or secrete it, and is targeted to tumor sites while exhibiting immune anti-cancer activity against tumors itself, and an anti-cancer composition comprising the same.

A further object of the present invention is to provide an anti-cancer adjuvant or tumor imaging adjuvant that can enhance the anti-cancer efficacy or imaging efficacy of labeled substances by targeting anti-cancer substances or contrast agents labeled with substances that specifically bind to Strep-tag expressed on the surface of the above strain and/or secreted, thereby directing them to the tumor site.

The present invention also aims to provide a method for providing information for cancer diagnosis utilizing the above tumor imaging adjuvant.

To achieve the aforementioned objectives, the present invention relates to a gene construct characterized by encoding a fusion protein of flgM and strep-tag through the linkage of an anti-sigma factor flgM gene and a gene encoding Strep-tag.

Unless otherwise defined, all scientific and technical terms used in this specification are defined to have the same meanings as commonly understood by those skilled in the art to which this invention belongs. Furthermore, in explaining the invention, detailed descriptions of known technologies related to the invention will be omitted if it determined that they may unnecessarily obscure the gist of the invention.

The fusion protein of flgM and strep-tag expressed by the gene construct of the present invention (hereinafter referred to as “flgM-Strep-tag”) was able to bind avidin or streptavidin similarly to Strep-tag, thereby demonstrating the biological equivalence of Strep-tag and flgM-Strep-tag. A special advantage of Strep-tag is that it is small in size and biochemically almost inactive. Therefore, unlike strains transformed to express biochemically active substances, which suffer reduced viability due to the accumulation of the expressed substances, expression of Strep-tag does not threaten strain survival, enabling stable expression. The gene construct of the present invention is characterized by being transformed into a strain to express flgM-Strep-tag on the surface of the strain and/or to secrete it.

In the gene construct of the present invention, the flgM gene and gene encoding Strep-tag may be directly linked or linked via a linker. The examples below exemplified constructs of SEQ ID NO: 1 or 2 connected via a linker having a gene sequence encoding an amino acid sequence consisting of GGSS-HIS*6 or GGGGS-HIS*6, but these are not limiting and those skilled in the art would readily modify the length or sequence of the linker appropriately.

To enhance expression efficiency, a Shine-Dalgarno sequence may be positioned upstream of the gene construct of the present invention. It is also evident that appropriate restriction enzyme sequences can be positioned at the upstream and downstream regions to facilitate the introduction of the gene construct into a vector.

The present invention further relates to a recombinant expression vector comprising the above gene construct. In the present invention, a “vector” refers to a DNA construct operably linked to enable the expression of the gene construct in a suitable host, which may be a plasmid, phage particle, or potential genomic insert. In the specification of the present invention, the terms plasmids and vectors are sometimes used interchangeably. It is natural that the expression vector can be selected and used with high efficiency according to the sequence of the gene construct.

In addition, the present invention relates to an anti-cancer recombinant strain that is transformed with the above gene construct operably linked to an inducible promoter, wherein the strain is characterized by expressing fusion protein flgM-Strep-5 tag on the surface of the strain, secreting flgM-Strep-tag or both expressing flgM-Strep-tag on the surface and secreting it, and targeting tumor cells. The strain of the present invention is known to exhibit immune anti-cancer activity by targeting tumors, and any strain that is harmless to humans can be used. Examples of such strains include E. coli, attenuated Salmonella, Shigella, Acinetobacter, Pseudomonas, Listeria, Clostridium or intestinal microorganisms (beneficial bacteria), probiotics, etc., but are not limited thereto.

The mere expression of flgM-Strep-tag by the strain cannot utilize the binding properties of Strep-tag. To utilize the binding between Strep-tag and Strep-tag-specific labeling substances, flgM-Strep-tag must either be expressed on the surface of the strain or secreted extracellularly. Expression of flgM-Strep-tag on the strain surface means that it is not secreted extracellularly but remains attached to the strain, for example through anchoring, while retaining its ability to bind to external substances. When flgM-Strep-tag is secreted, the secreted substance may migrate or diffuse elsewhere, reducing its targeting ability, so expression on the surface of the strain is more desirable. The recombinant strain of the present invention can be transformed with a recombinant expression vector comprising the above gene construct. The transformation method follows conventional techniques and can use, for example, calcium chloride method, electroporation, etc., but is not limited thereto. The recombinant strain of the present invention is characterized by not only expressing flgM-Strep-tag encoded by the gene construct of the present invention simply inside cells by the operation of flgM, but also expressing it on the surface and/or secreting it extracellularly. As described above, since Strep-tag is almost biochemically inactive, the strain is not killed by GM-CSF expressed inside the cell and can stably and continuously express/secrete fusion proteins.

In this specification, “operably linked to an inducible promoter” refers to the ability to switch the operation of the linked gene using an inducing substance. The inducible promoter may be, for example, tac promoter, lac promoter, lacUV5 promoter, lpp promoter, pLA promoter, pRA promoter, rac5 promoter, amp promoter, recA promoter, SP6 promoter, trp promoter, T7 promoter, pBAD promoter, Tet promoter, trc promoter, pepT promoter, sulA promoter, pol II(dinA) promoter, ruv promoter, uvrA promoter, uvrB promoter, uvrD promoter, umuDC promoter, lexA promoter, cea promoter, caa promoter, recN promoter, pagC promoter, hip promoter, ansB promoter, or pflE promoter.

In particular, the strain of the present invention may be an attenuated Salmonella strain. Unlike other strains, Salmonella strains can infiltrate the liver and spleen to stimulate systemic immune responses, so their immune anti-cancer activity is even more excellent. “Attenuation” refers to the artificial weakening of a living pathogen's toxicity by mutating genes involved in essential metabolism so that it cannot cause disease in the body and only stimulates the immune system to induce immunity. Salmonella attenuation-inducing genes are well known in the art and can be achieved by loss of function of one or more genes selected from the group consisting of aroA, aroC, aroD, aroE, Rpur, htrA, ompR, ompF, ompC, galE, cya, crp, cyp, phoP, phoQ, rfaY, dksA, hupA, sipC, clpB, clpP, clpX, pab, nadA, pncB, pmi, rpsL, hemA, rfc, poxA, galU, cdt, pur, ssa, guaA, guaB, fliD, flgK, flgL, relA and spor. To prevent reversion to wild-type during in vivo application and further weaken toxicity, the functions of two or more of the above genes may be disrupted.

In particular, when flgM-Strep-tag is expressed in attenuated Salmonella strains, it was observed that flagella disappeared resulting in inhibited motility or even complete loss of motility. This indicates that safety is significantly improved because movement to other organs is restricted by expressing flgM-Strep-tag after targeting the tumor site. Specifically, the strain may be S-flgM-streptag-pBAD18-ASD+/BRD509 asd-with deposit number KCTC15484BP, deposited at the Korean Collection for Type Cultures of the Korea Research Institute of Bioscience and Biotechnology on Jun. 26, 2023, but is not limited thereto.

The present invention further relates to an anti-cancer composition comprising the above anti-cancer recombinant strain as an active ingredient. The anti-cancer recombinant strain of the present invention can be used as an anti-cancer composition because it naturally targets tumor sites itself and exhibits immune anti-cancer activity.

The composition of the present invention can be prepared using established methods in the pharmaceutical field for use as an anti-cancer agent, and can be used alone or mixed with pharmaceutically acceptable carriers, forming agents, diluents, etc.

The composition of the present invention can be formulated into preparations for oral or parenteral administration. The dosage of the active ingredient according to the present invention can be appropriately adjusted according to absorption of active ingredients in vivo, formulation type, age, gender and condition of patients, degree of symptoms, etc., and can be administered once a day or divided into multiple doses. The typical dosage ranges from 0.001 mg/kg·day to 10g/kg·day.

In addition, the present invention relates to an anti-cancer adjuvant characterized by comprising the above anti-cancer recombinant strain as an active ingredient and being co-administered with an anti-cancer agent labeled with a substance that specifically binds to Strep-tag. Examples of substances that specifically bind to Strep-tag include avidin, streptavidin, strep-tactin, but are not limited thereto. The strain of the present invention, after targeting tumor cells, can express Strep-tag on its surface and/or secrete it in the form of an flgM-Strep-tag fusion protein in respons to an inducing substance, thereby enabling the targeted delivery of anti-cancer agents labeled with substances that specifically bind to strep-tag to the tumor. Therefore, side effects of anti-cancer agents on other organs can be minimized and anti-cancer activity can be enhanced, and anti-cancer activity can be shown even at lower doses, reducing the burden on patients caused by side effects. As the anti-cancer agent, any substance that has little or no ability to target tumor cells itself while exhibiting anti-cancer activity can be used as long as it can be labeled with substances that specifically bind to Strep-tag, such as nanoparticles, antibodies, anti-cancer proteins or small molecule substances.

The anti-cancer recombinant strain of the present invention can also be utilized as a tumor imaging adjuvant that can enhance tumor tissue imaging efficacy by co-administration with contrast agents labeled with substances that specifically bind to Strep-tag, thereby targeting the contrast agent to tumor sites. As the contrast agent, one or more selected from the group consisting of radioactive nuclides, fluorescent labels, enzyme labels, chemiluminescent markers, gold preparations and magnetic preparations can be used, and determining the imaging method based on the type of contrast agent would be easy for those skilled in the art. The tumor imaging adjuvant of the present invention not only enhances tumor imaging efficiency but also exhibits anti-cancer activity itself, so it can have more beneficial effects for cancer patients.

The tumor imaging adjuvant of the present invention can be effectovely used for providing information for cancer diagnosis. For example, providing information for cancer diagnosis can be carried out by a method comprising: (A) targeting tumor cells by administering the tumor imaging adjuvant of the present invention to a patient suspected of having cancer or undergoing anti-cancer treatment; (B) inducing expression of flgM and Strep-tag fusion protein in the recombinant strain of the tumor imaging adjuvant; (C) administering a contrast agent labeled with a substance that specifically binds to Strep-tag; and (D) detecting and imaging the contrast agent. However, it is not limited to the above steps, and appropriate modifications can naturally be made based on the type and characteristics of the strain and contrast agent, for instance, steps (B) and (C) may be carried out simultaneously, or steps (A) and (C) may be carried out simultaneously, or additional steps may be added.

As described above, the gene construct of the present invention, when introduced into an anti-cancer strain that targets tumor cells and shows immune anti-cancer activity, allows the strain to express flgM-strep-tag on its surface and/or secrete it, which has biologically equivalent function to Strep-tag.

Therefore, the anti-cancer recombinant strain introduced with the gene construct of the present invention can not only exhibit anti-cancer activity itself but also enhance anti-cancer adjuvant or tumor imaging adjuvant by targeting anti-cancer agents or contrast agents to tumors when co-administered with anti-cancer agents or contrast agents that have no tumor targeting function but are labeled with substances that specifically bind to Strep-tag.

### BRIEF DESCRIPTION OF THE DRAWINGS

FIG. 1 is a diagram showing DNA base sequences designed for preparing plasmids for expression of flgM-Strep-tag fusion protein in one embodiment.

FIG. 2 is a diagram showing DNA base sequences designed for preparing plasmids for expression of flgM-Strep-tag fusion protein in another embodiment.

FIG. 3 is an electrophoresis photograph quantifying flgM-Strep-tag in the culture medium of strains transformed with the above plasmids.

FIG. 4 is a SEM image and quantification graphs showing that Strep-tag was expressed on the surface of a strain in one embodiment of the present invention.

FIG. 5 is fluorescence images and histograms showing that Strep-tag was expressed by a strain in one embodiment of the present invention.

FIG. 6 is images showing inhibition of strain motility by induction of flgM-Strep-tag fusion protein expression.

### DETAILED DESCRIPTION

The present invention is described in more detail below with reference to the accompanying drawings and examples. However, these drawings and examples are only illustrative to easily explain the content and scope of the technical idea of the present invention, and the technical scope of the present invention is not limited or altered thereby. Based on such examples, various modifications and changes are possible within the scope of the technical idea of the present invention, which would be obvious to those skilled in the art.

### EXAMPLES

### Example 1: Construction of Plasmids for Expression of Strep-Tag

Plasmids were constructed by fusing flgM protein, which is secreted extracellularly through the basal body and hook, with Strep-tag.

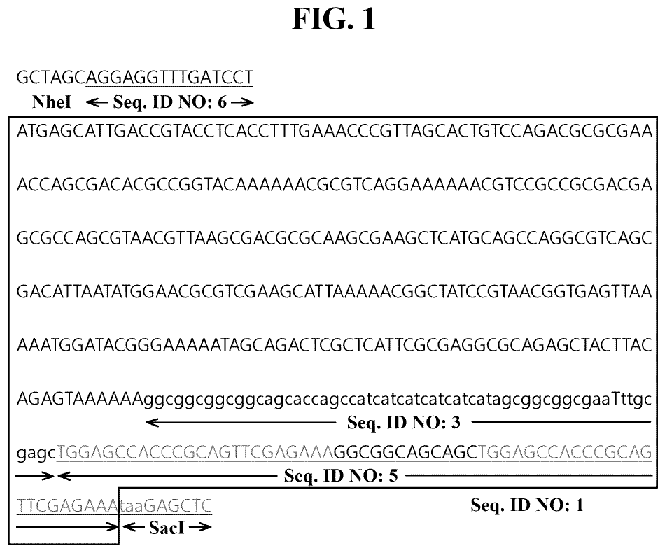

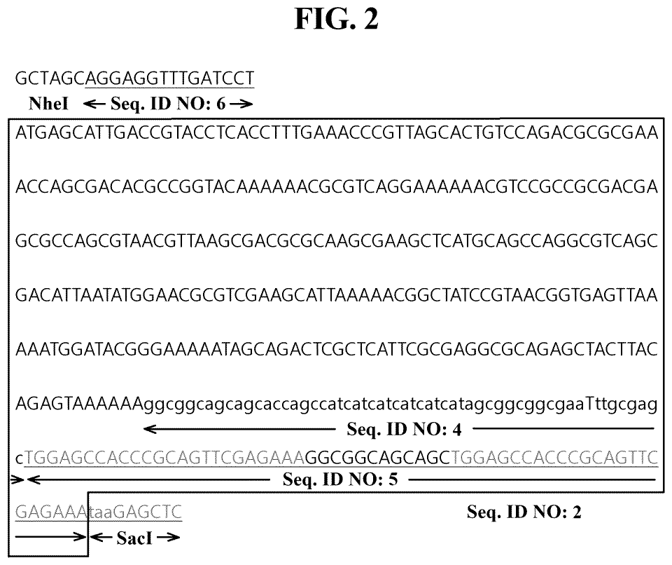

First, genes capable of expressing fusion proteins of flgM and Strep-tag were designed. The DNA sequence of wild-type Salmonella flgM (flgM anti-sigma-28 factor FlgM [Salmonella enterica subsp. enterica serovar Typhimurium str. LT2] Gene ID: 1252690, Locus tagSTM1172 NC_003197.2 (1257036..1257341, complement)) and the DNA sequence obtained by Salmonella codon-optimizing the amino acid sequence of Strep-tag (Nature Protocols volume 2, pages 1528-1535 (2007)) were combined via a linker. For the linker (Seq. ID NO: 3 or Seq. ID NO: 4), either a GGGGS linker or GGSS linker, connected with 6 histidines, was used, and Strep-tag was configured to express a twin Strep-tag (Seq. ID NO: 5) wherein two Strep-tags are connected by GGSS linker. To increase expression of the flgM gene, Shine-Dalgarno sequences (Seq. ID NO: 6) were inserted in front of the flgM gene, and for cloning, NheI site was inserted at the very front, and SacI site was inserted after the stop codon of flag tag. FIGS. 1 and 2 show the base sequence of flgM-GGGGS-HIS*6-streptag-GGSS-streptag and flgM-GGSS-HIS*6-streptag-GGSS-streptag designed through the above process. The gene sequences were synthesized by Cosmogenetech Co., Ltd. The sequences excluding the restriction enzyme sites and shine dalgarno sequences correspond to genes encoding the fusion proteins of flgM and Strep-tag (SEQ ID NO: 1 and SEQ ID NO: 2).

The synthesized flgM-GGGGS-HIS*6-streptag-GGSS-streptag and flgM-GGSS-HIS*6-streptag-GGSS-streptag were digested with 10 U each of NheI (Takara) and SacI (Takara) at 37° C. for 2 hours. The reaction products were electrophoresed and 400 bp DNA was extracted using a Gel extraction kit (Qiagen) to isolate insert DNA for each fusion protein flgM-Strep-tag.

pBad18 asd plasmid (obtained from Prof. Heon-Man Lim's lab at Chungnam National University) was digested with 100 each of NheI and SacI enzymes at 37° C. for 2 hours, and the reaction product was purified using a PCR purification kit (Qiagen) to obtain vector DNA. The vector DNA was ligated (Invitron) with the two flgM-Strep-tag inserts at 25° C. for 30 minutes each and transformed into DH5a competent cells. The transformed cells were plated on LB amp solid medium and cultured at 37° C. to select colonies with antibiotic resistance. 6 candidates were selected from each selected colony and digested with 10 U each of Nhel and SacI at 37° C. for 1 hour, and the formation of 400 bp bands was confirmed by electrophoresis. The nucleotide sequences of the candidates were analyzed by Cosmogenetech Co., Ltd. to confirm the insertion of genes of SEQ ID NO: 1 or 2, and flgM-GGGGS-HIS*6-streptag-GGSS-streptag plasmid and flgM-GGSS-HIS*6-streptag-GGSS-streptag plasmid were successfully constructed for secretion of flgM-Strep-tag.

### Example 2: Preparation of Attenuated Salmonella Strain Expressing flgM-Strep Tag Fusion Protein

### 1) Transformation of Attenuated Salmonella Strain and Confirmation of flgM-Strep Tag Fusion Protein Secretion

Using each of the flgM-GGGGS-HIS*6-streptag-GGSS-streptag plasmid and flgM-GGSS-HIS*6-streptag-GGSS-streptag plasmid prepared in Example 1, attenuated Salmonella strains were individually transformed, and expression of flgM-Strep tag fusion protein was examined in the culture medium to confirm secretion of the fusion protein. For the attenuated Salmonella strain, aroA aroD asd-strain (obtained from Prof. Heon-Man Lim at Chungnam National University) was used.

A single colony of each transformed strain was inoculated into LB amp liquid medium and shaken cultured at 37° C. for 12-16 hours. Subsequently, it was diluted with fresh LB amp liquid medium to OD₆₀₀ of 0.05 and shaken cultured at 37° C. for 2 hours. When OD₆₀₀ reached 0.4-0.6 by shaking culture, arabinose was treated to a final concentration of 0.2 w %, and shaken cultured at 37° C. for an additional 5 hours. For comparison, the control group was shaken cultured under the same conditions without arabinose treatment. The culture medium was centrifuged at 3000 rpm to separate the supernatant, and filtered through a 0.2 um filter to completely remove bacteria.

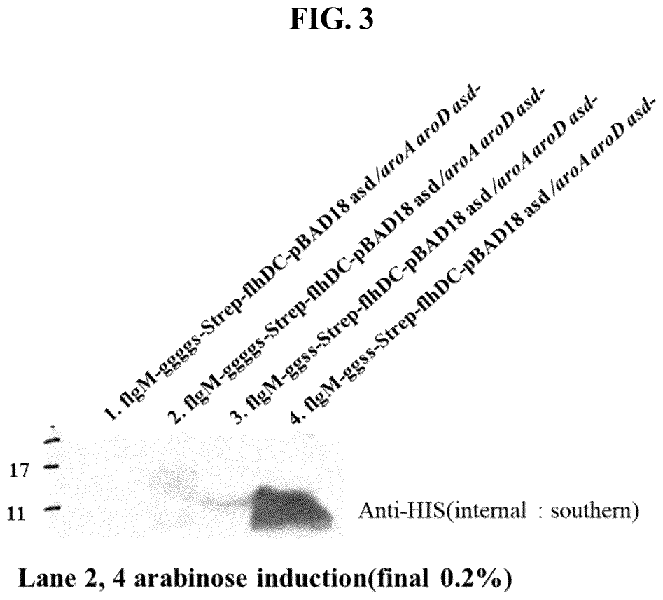

The amount of flgM-Strep-tag in the culture medium was confirmed through Western blot from the bacteria-free supernatant. FIG. 3 is an electrophoresis photograph showing the detection results of the fusion protein in the culture medium of experimental groups, wherein lanes 1 and 3 are from the experimental groups not treated with arabinose, and lanes 2 and 4 are from the experimental groups treated with arabinose. In FIG. 3, both strains showed detection of fusion proteins in the culture medium when treated with arabinose. In addition, the Salmonella strain transformed by the flgM-GGSS-HIS*6-streptag-GGSS-streptag plasmid showed remarkably superior flgM-Strep-tag fusion protein secretion ability compared to the Salmonella strain transformed by the flgM-GGGGS-HIS*6-streptag-GGSS-streptag plasmid. Accordingly, the strain transformed by the flgM-GGSS-HIS*6-streptag-GGSS-streptag plasmid was named S-flgM-streptag-pBAD18-ASD+/BRD509 asd-and deposited at the Korean Collection for Type Cultures of the Korea Research Institute of Bioscience and Biotechnology on Jun. 26, 2023 (deposit number KCTC15484BP).

Below, the efficacy resulting from flgM-Strep-tag fusion protein expression was tested using the S-flgM-streptag-pBAD18-ASD+/BRD509 asd-strain.

### 2) Testing Expression of Strep-Tag on Surface of Salmonella Strain

A single colony of the attenuated Salmonella strain expressing the flgM-Strep-tag fusion protein prepared in 1) above was put into 20 mL of LB amp liquid medium and cultured at 37° C. for 16 hours. 200 μL of the above culture was diluted into 20 mL of fresh LB amp liquid medium, then further cultured for an additional 2 hours at 37° C. until OD₆₀₀ reached 0.4-0.6. Subsequently, 200 pL of 20% (w/v) arabinose was added to achieve a final concentration of 0.2% and culture was incubated at 37° C. for 6 hours. The same amount of PBS was added to the control group. After 6 hours of culture, 2 mL of the culture sample with OD_{600 1.0} was taken and washed 3 times with medium (centrifugation at 4200 rpm for 5 minutes) to prepare 1.0 mL of a bacterial sample with OD₆₀₀ 1.0.

Separately, 0.25 g of streptavidin-coated nanoparticles with a 200 nm diameter (provided by Prof. Jin-Sil Choi at Hanbat National University) was suspended in 1 mL of PBS to prepare a nanoparticle solution.

50 μL of the bacterial sample prepared above and 50 μL of the nanoparticle solution were mixed at 600 rpm using a vortex mixer at room temperature for 30 minutes. After 30 minutes, the mixture was centrifuged at 4200 rpm for 5 minutes, and the supernatant was removed. Then, 0.1 mL of 4% glutaraldehyde was added and mixed, and the mixture was left in the refrigerator overnight for fixation.

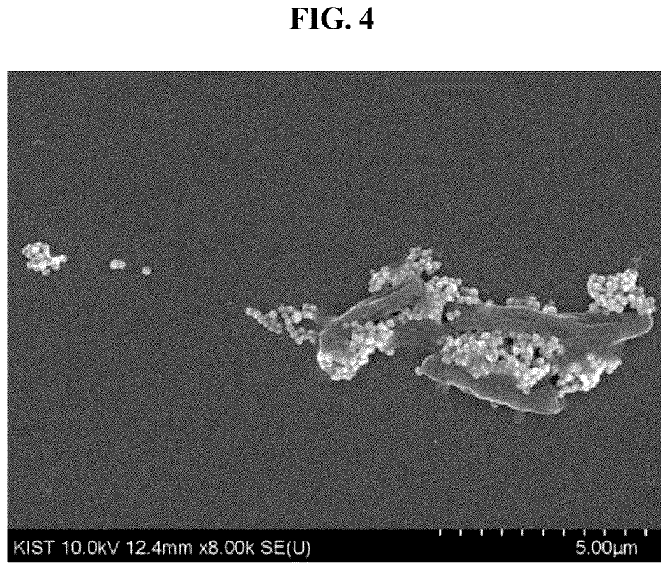

4 μL of the fixed bacterial solution was dropped onto a wafer and dried for 2 hours in vacuum at 37° C., then platinum coated for 20 seconds at 10 mA and observed with FE-SEM (Regulus8230). FIG. 4 shows SEM images taken where nanoparticles were observed to be attached to the surface of bacteria in a cluster form. This is thought to be because Strep-tag is expressed on the bacterial surface, nanoparticles attach to the surface of the strain through binding between expressed surface Strep-tag on the and streptavidin of the nanoparticles, and Strep-tag secreted from the strain acts as a crosslinker causing clustering of streptavidin-coated nanoparticles. In contrast, nanoparticles attached to the Salmonella surface were not observed in the control group (data not shown).

### 3) Testing Expression of Strep-Tag by Fluorescence Analysis

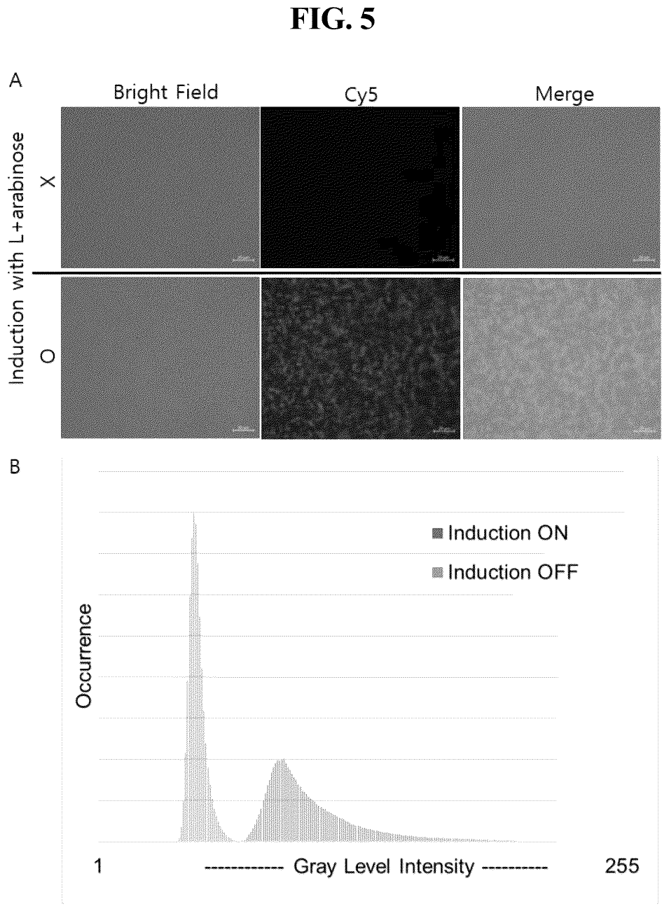

Expression of Strep-tag was additionally confirmed by observing Cy5 fluorescence signals using Streptavidin, flamma 648(BioActs) dye. For this, 170 μL of bacterial sample with OD₆₀₀ 1.0, prepared by the same method as the experiment using nanoparticles above, 20 μL of PBS, and 10 μL of 100 μg/mL dye solution were mixed using a vortex mixer at 500 rpm under dark conditions. To remove unbound dye from the Salmonella strain, the mixture was centrifuged at 8000 rpm for 10 minutes after 1 hour, the supernatant was removed, then the pellet was resuspended in 200 μL of motility medium and photographed with a fluorescence microscope. FIG. 5A shows bright field microscope images and fluorescence images, and FIG. 5B shows histograms comparing the average gray level intensity values between the presence and absence of arabinose induction after converting RGB values to gray scale. As can be seen in FIG. 5A, when arabinose was not added, Cy5 fluorescence was barely observed, but when Strep-tag expression was induced by arabinose, strong Cy5 fluorescence was observed, confirming that Strep-tag was expressed in the Salmonella strain. The histogram in FIG. 5B also clearly shows that when Strep-tag expression was induced by arabinose, the gray level intensity was observed in brighter parts compared to the group without arabinose added.

### 4) Testing Motility of Salmonella Strain

As the flagella of the Salmonella strain were not observed in the SEM images of FIG. 4, motility was tested to determine whether the induction of Strep-tag expression actually affects the motility of Salmonella.

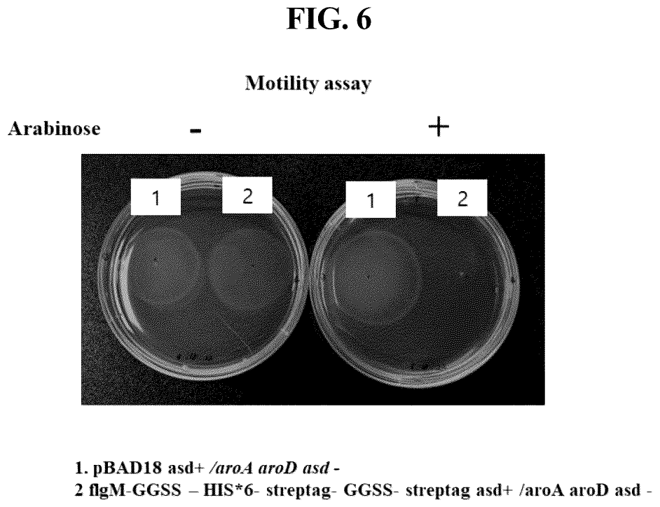

Motility testing was performed using soft agar tryptone plates (per liter: 10 g Bacto tryptone (BD: 211705), 5 g NaCl (Biosesang S1009), and 3 g Bacto agar (BD: 214010)). Single colonies of (1) the Salmonella strain transformed with pBAD18 asd+/aroA aroD asd-plasmid and (2) the Salmonella strain transformed with flgM-GGSS-HIS*6-streptag-GGSS-streptag plasmid prepared in Example 2 were each transplanted at separated positions using sterilized toothpicks onto soft agar tryptone plates with or without 0.2% arabinose. Subsequently, motility was confirmed after culturing at 37° C. for 5 hours. FIG. 6 shows the results, demonstrating that the motility of the Salmonella strains was lost by expression of flgM-Strep-tag fusion protein.
Appendix to the Specification: Receipt in the case of an original deposit
Name of depositor: The Korean Collection for Type Cultures, Deposit date: Jun. 26, 2023, Accession number: KCTC15484BP, the address of the Korean Collection for Type Cultures is Korea Research Institute of Bioscience and Biotechnology (KRIBB) 181, Ipsin-gil, Jeongeup-si, Jeolllabuk-do, 56212, Republic of Korea. The deposit was made under Budapest Treaty, and that all restrictions imposed by the depository will be irrevocably removed upon the granting of the patent. The deposit is hereby incorporated by reference in its entirety.

1. A gene construct characterized by encoding a fusion protein of flgM and Strep-tag through the linkage of an anti-sigma factor flgM gene and a gene encoding Strep-tag.

2. The gene construct according to claim 1,
wherein the flgM gene and gene encoding strep-tag are directly linked or linked via a linker.

3. The gene construct according to claim 1,
wherein it consists of SEQ ID NO: 1 or SEQ ID NO: 2.

4. A recombinant expression vector comprising the gene construct of claim 1.

5. An anti-cancer recombinant strain that is transformed with the gene construct of claim 1 operably linked to an inducible promoter, wherein the strain is characterized by expressing fusion protein of flgM and Strep-tag on the surface of the strain, secreting fusion protein of flgM and Strep-tag, or both expressing fusion protein of flgM and Strep-tag on the surface of the strain and secreting it, and targeting tumor cells.

6. The anti-cancer recombinant strain according to claim 5, wherein the strain is an attenuated Salmonella strain.

7. The anti-cancer recombinant strain according to claim 6,
wherein the strain has inhibited or lost motility when expressing fusion protein of flgM and strep-tag by the inducible promoter.

8. The anti-cancer recombinant strain according to claim 7,
wherein the strain is the strain with deposit number KCTC15484BP.

9. A method of treating a cancer, comprising:
administering an anti-cancer composition comprising the anti-cancer recombinant strain of claim 5 to a subject.

10. An anti-cancer adjuvant characterized by comprising the recombinant strain of claim 5 and being co-administered with an anti-cancer agent labeled with a substance that specifically binds to Strep-tag.

11. The anti-cancer adjuvant according to claim 10,
wherein the substance that specifically binds to Strep-tag is avidin, streptavidin or Strep-tactin.

12. A tumor imaging adjuvant characterized by comprising the recombinant strain of claim 5 and being co-administered with a contrast agent labeled with a substance that specifically binds to Strep-tag.

13. The tumor imaging adjuvant according to claim 12,
wherein the substance that specifically binds to Strep-tag is avidin, streptavidin or Strep-tactin.

14. The tumor imaging adjuvant according to claim 12,
wherein the contrast agent is one or more selected from the group consisting of radioactive nuclides, fluorescent labels, enzyme labels, chemiluminescent markers, gold preparations and magnetic preparations.

15. A method for providing information for cancer diagnosis comprising:
(A) targeting tumor cells by administering the tumor imaging adjuvant of claim 14 to a patient suspected of having cancer or undergoing anti-cancer treatment;
(B) inducing expression of flgM and strep-tag fusion protein in the recombinant strain of the tumor imaging adjuvant;
(C) administering a contrast agent labeled with a substance that specifically binds to Strep-tag; and
(D) detecting and imaging the contrast agent.
